(19) United States
(12) Patent Application Publication
(10) Pub. No.: US 2002/0040013 A1
(43) Pub. Date: Apr. 4, 2002

(54) Novel therapeutic use of low molecular weight heparins

(76) Inventors: Jean-Marie Stutzmann, Villecresnes (FR); Andre Uzan, Paris (FR)

Correspondence Address: AVENTIS PHARMACEUTICALS, INC., PATENTS DEPARTMENT, ROUTE 202-206, P.O. BOX 6800, BRIDGEWATER, NJ, 08807-0800, US

(21) Appl. No.: 09/881,267

(22) Filed: Jun. 14, 2001

Related U.S. Application Data
(63) Continuation of application No. PCT/FR99/03109, filed on Dec. 13, 1999.

(30) Foreign Application Priority Data
Dec. 17, 1998 (FR) 98/15919

(51) Int. Cl. A61K 31/727; C08B 37/10

(52) U.S. Cl. 514/56; 536/21

(57) ABSTRACT

The invention concerns the use of low molecular weight heparin for preventing and/or treating motor neuron diseases.

The present invention relates to the use of low molecular weight heparins in the prevention and/or treatment of motoneuron diseases.

Standard heparin is a sulfated polysaccharide with a mean molecular weight of 12 000-15 000 daltons, isolated from the intestinal mucous membranes of cattle, sheep and pigs. Heparin is used clinically for the prevention and treatment of thromboembolic disorders, but sometimes causes hemorrhages.

For the past ten or so years, heparin has been progressively replaced with low molecular weight heparins which no longer exhibit, or exhibit to a lesser degree, the drawback of causing bleeding, and which now require only one injection per day instead of 2 to 3 injections per day for standard heparin. These low molecular weight heparins are prepared, in particular, by fractionation or controlled depolymerization of heparin, or by chemical synthesis. They have an anti-Xa activity/anti-IIa activity ratio of greater than 2.

It has now been found that low molecular weight heparins increase the survival and/or growth of motoneurons and can thus be used in the prevention and/or treatment of motoneuron diseases.

Motoneuron diseases include amyotrophic lateral sclerosis, progressive spinal muscular atrophy, infantile muscular atrophy and primary lateral sclerosis.

According to the invention, a low molecular weight heparin having a mean molecular weight of between 1 000 and 10 000 daltons, especially between 1 500 and 6 000 daltons, and in particular between 4 000 and 5 000 daltons, is used.

They can be prepared using different processes, from heparin:

fractionation using solvents (FR2440376, US 4692435),

fractionation on anionic resin (FR2453875),

gel filtration (Barrowcliffe, Thromb. Res. 12, 27-36 (1977),

affinity chromatography (US4401758),

controlled depolymerization using a chemical agent: nitrous acid (EP14184, EP37319, EP76279, EP623639, FR2503714, US4804652, WO813276), □-elimination using a heparin ester (EP40144, US5389618), periodate (EP287477), sodium borohydride (EP347588, EP380943), ascorbic acid (US4533549); hydrogen peroxide (US4629699, US4791195), quaternary ammonium hydroxide using a quaternary ammonium salt of heparin (US4981955), alkali metal hydroxide (EP380943, EP347588), or by enzymatic process (EP64452, US4396762, EP244235, EP244236; US 4826827; US 3766167) or using irradiation (EP 269981).

Some can also be prepared by chemical synthesis (US4801583, US4818816, EP165134, EP84999, FR2535306).

Among these low molecular weight heparins, mention may be made more particularly of enoxaparin (INN) sold by Rhone-Poulenc Rorer, nadroparin (INN) sold by Sanofi, parnaparin (INN) sold by Opocrin-alfa, reviparin (INN) sold by Knoll, dalteparin (INN) sold by Kabi Pharmacia, tinzaparin (INN) sold by Novo Nordisk, danaparoid (INN) sold by Organon, ardeparin (INN) developed by Wyeth Ayerst, certoparin (INN) sold by Sandoz and products being studied, such as CY222 from Sanofi-Choay (Thromb. Haemostasis, 58 (1), 553 (1987)) or SR90107/ORG31540 from Sanofi-Organon (Thrombosis and Haemostasis, 74,1468-1473 (1995)).

Preferably, the low molecular weight heparins consist of oligosaccharides having a 2-O-sulfo-4-enopyranosuronic acid at one of their ends.

A particularly advantageous low molecular weight heparin is obtained by depolymerization of a heparin ester and, in particular, a benzyl ester, using a base such as sodium hydroxide.

In the presence of trophic support provided by the neurotrophic factors BDNF or NT5, motoneuron cultures are composed of large and homogeneous neurons with long branched neurites. However, the motoneurons die by apoptosis if the culture is carried out in the absence of trophic support.

The effect of low molecular weight heparins was therefore determined in a model of degeneration induced by starving motoneurons in culture of neurotrophic factors.

In addition, astrocytes play a major role in the control and maintenance of a suitable environment for motoneuron survival.

The effect of low molecular weight heparins was thus also tested on a coculture of motoneurons and astrocytes.

The protocols used are as follows:

Cultures Enriched in Motoneurons:

The cultures enriched in motoneurons are prepared using the centrifugation method described by R. L. Schnaar and A. E. Schaffner, J. Neurosci., 1, 204-217 (1981) and modified byW. Camu and C. E. Henderson, J. Neurosci. Methods, 44, 59-70 (1992). Spinal cords from E15 rat embryos are dissected sterilely and the spinal notochords are removed. They are then cut up and incubated for 15 minutes at 37° C. in PBS (phosphate buffered saline: 137 mM NaCl, 2.68 mM KCl, 6.45 mM Na₂HPO₄, 1.47 mM KH₂PO₄) to which 0.05% of trypsin has been added. The dissociation of the cells is completed by trituration with the end of a 1 ml pipette in the culture medium supplemented with bovine serum albumin (BSA) and with DNAase. The cell suspension is spread onto a band of 6.5% weight/volume metrizamide in L15 medium (sold by Gibco BRL) and centrifuged at 500 g for 15 minutes. The band of the interface containing the motoneurons is recovered. The motoneurons are plated out at a density of 5 000 cells per 35 mm in culture dishes precoated with polyornithine-laminin in an L15 medium to which sodium bicarbonate (22 mM), coalbumin (0.1 mg/ml), putrescine (0.1 mM), insulin (5 □g/ml), sodium selenite (31 nM), glucose (20 mM), progesterone (21 nM), penicillin (100 lU/ml) and streptomycin (100 ug/ml) have been added. The cultures are maintained at 37° C. in a humidified atmosphere at 5% CO₂.

Culturing of Spinal Cord Astrocytes:

The astrocytes are obtained from rat embryos according to the method of R. P. Saneto and J. de Vellis, in Neurochemistry, a practical approach (A. J. Turner and H. S. St John) IRL Press, Oxford-Washington DC, p27-63 (1987), slightly modified. The spinal cords are dissected sterilely, and the meninges and dorsal ganglia are removed. Five to ten spinal cords are transferred into PBS (phosphate buffered saline: 137 mM NaCl, 2.68 mM KCl, 6.45 mM Na₂HPO₄, 1.47 mM KH₂PO₄) and cut up before incubation at 37° C. for 25 minutes in PBS to which 0.25% of trypsin has been added. The enzymatic treatment is stopped by adding 10 ml of Dubelcco modified Eagle medium (DMEM) to which 10% of fetal calf serum (FCS) has been added, and the cells are collected by centrifugation. Another step of mechanical dissociation is carried out using the end of a 1 ml pipette. The cells are plated out at a density of 1.2-2 □10⁶ cells per 25 cm² of culture medium in DMEM containing 10% of FCS. After 2 days in vitro, the cultures are fed each day throughout the duration of the study. When a visible monolayer of cells is obtained, the cultures are shaken for 48 hours at 250 rpm and, the following day, the monolayers are treated with cytosine arabinoside (10⁻⁵ M) for 48 hours. The monolayers of astrocytes are then amplified at a density of five for 35 mm on culture plates for 25 cm² culture flasks at the start of the study.

The cultures of spinal astrocytes are composed of more than 98% cells which are immunoreactive for glial fibrillary acidic protein (GFAP). The monolayers of astrocytes are exposed to the product to be tested in solution in water for 24 hours at the concentration indicated. The monolayers of astrocytes are then washed with DMEM and maintained for 2 hours with culture medium to which the motoneurons have been added. Two hours after feeding, and for 2 or 3 days, the vehicle or product to be tested is again added to the culture medium.

Immunochemistry

The cells are fixed in 4% paraformaldehyde and 0.1% glutaraldehyde in PBS (pH 7.4 at 4° C. for 15 minutes). The cultures are then washed and the nonspecific sites are blocked with 10% of goat serum and 2% of bovine serum albumin (BSA) in PBS. These cultures are successively incubated with Islet ½ transcription factor antibodies overnight at 4° C. and streptavidin-peroxidase antibodies (1/200, Gibco) for 60 minutes. The antibodies are visualized using the DAB/hydrogen peroxide reaction. Antineurofilament antibodies (LC Amersham) are used to identify neurites.

Cell Counting and Statistical Analysis

The cells which are immunoreactive for the Islet ½ homoprotein or for neurofilaments, and which exhibit neurites longer than the diameters of 10 cells, are considered to be viable motoneurons. The number of motoneurons is evaluated by counting labeled cells in a surface area of 1.44 cm² under a microscope giving a 200-fold magnification. The values are expressed as a number of motoneurons per cm² or a percentage of the number of motoneurons present in the cultures maintained with trophic factors (BDNF/NT5 1 ng/mg). The experiments are carried out at least 3 times.

The statistical analyses are carried out using the Student's test (t-test).

The assays were carried out using enoxaparin as the low molecular weight heparin.

The results obtained are as follows:

1—Effect of Various Concentrations of Enoxaparin on the Number of Motoneurons in the Astrocyte-Motoneuron Cocultures

| | Number of motoneurons |
|---|---|
| | % with respect |
| | to the control ± |
| | standard deviation |
| Vehicle | 100 ± 21 |
| Enoxaparin | |
| 1 ng | 118 ± 33 |
| 10 ng | 196 ± 47 |
| | (P < 0.05) |
| 50 ng | 149 ± 22 |

These results demonstrate that pretreating the astrocytes with enoxaparin increases the number of motoneurons which grow on the monolayer of astrocytes.

In this test, the inoxaparin induces no apparent morphological effect.

2—Effect on Motoneuron Survival in the Astrocyte-Motoneuron Cocultures

| | Motoneuron survival |
|---|---|
| | % with respect |
| | to the control ± |
| | standard deviation |
| Vehicle | 99.9 ± 5.1 |
| Enoxaparin | |
| 1 ng/ml | 109.3 ± 16.9 |
| 10 ng/ml | 120.7 ± 3.2 |
| | (P = 0.0066) |

These results show that enoxaparin increases the survival of motoneurons.

3—Effect on the Number of Very Large Motoneurons

| | Number of large motoneurons |
|---|---|
| | (500 □m) per cm³ |
| Vehicle | 38 |
| Enoxaparin | |
| 1 ng/ml | 48 |
| 10 ng/ml | 66 |

The results demonstrate that enoxaparin increases the number of large motoneurons with respect to the control.

4—Potentiation Effect on the Stimulation of the Trophic Motoneuron Activity

Monolayers of astrocytes respond to the stress induced by exposure to sublethal concentrations of free radicals and increases the production of the trophic activity of motoneurons. In particular, fluxes of low concentrations of peroxinitrite formed by SIN-1 (200 umol/min) considerably stimulate the trophic capacity of monolayers of astrocytes once the stimulus has ended. The effect of enoxaparin on this effect was therefore studied.

The monolayers of astrocytes are treated for 24 hours with the vehicle or the enoxaparin (10 ng/ml), and are treated for 1 hour with 2 mM of SIN-1 (nitrogen-containing medium). After washing, the motoneurons are plated out in L15 medium. After 2 hours, the vehicle or inoxaparin is added to the culture media once again.

| | Number of motoneurons |
|---|---|
| | % with respect to the control |
| Vehicle | 100 |
| SIN-1 (2 mM) | 125 |
| Enoxaparin (10 ng/ml) | 115 |
| Enoxaparin (10 ng/ml) + | 160 |
| SIN-1 (2 mM) | |

These results demonstrate that the enoxaparin and SIN-1 increase the trophic capacity of the astrocytes. Moreover, the enoxaparin potentiates the trophic effect of the SIN-1.

The present invention relates to the use of a low molecular weight heparin for preparing a medicinal product which is useful for the survival and/or growth of motoneurons.

The present invention also relates to a low molecular weight heparin for preparing a medicinal product which is useful in the prevention and/or treatment of motoneuron diseases, and in particular amyotrophic lateral sclerosis, progressive spinal muscular atrophy, infantile muscular atrophy and primary lateral sclerosis.

The medicinal products consist of a salt (sodium or calcium preferably) or a low molecular weight heparin in the form of a composition in which the salt is combined with any other pharmaceutically compatible product, which may be inert or physiologically active. The medicinal products according to the invention can be used intravenously, subcutaneously, orally, rectally, topically or via the pulmonary route (inhalation).

The sterile compositions for intravenous or subcutaneous administration are generally aqueous solutions. These compositions may also contain adjuvants, in particular wetting agents, tonicity agents, emulsifiers, dispersing agents and stabilizers. The sterilization can take place in several ways, for example by aseptic filtration, by incorporating sterilizing agents into the composition, or by irradiation. They may also be prepared in the form of sterile solid compositions which can be dissolved at the time of use in sterile water or any other injectable sterile medium.

As solid compositions for oral administration, it is possible to use tablets, pills, powders (gelatin capsules, cachets) or granules. In these compositions, the active principle is mixed with one or more inert diluents, such as starch, cellulose, sucrose, lactose or silica, under a stream of argon. These compositions may also comprise substances other than diluents, for example one or more lubricants, such as magnesium stearate or talc, an agent which promotes oral absorption, a colorant, a coating (dragees) or a varnish.

As liquid compositions for oral administration, it is possible to use solutions, suspensions, emulsions, syrups and elixirs which are pharmaceutically acceptable, containing inert diluents such as water, ethanol, glycerol, plant oils or paraffin oil. These compositions may comprise substances other than diluents, for example wetting products, sweeteners, thickeners, flavorings or stabilizers.

The compositions for rectal administration are suppositories or rectal capsules which contain, besides the active product, excipients such as cocoa butter, semi-synthetic glycerides or polyethylene glycols.

The compositions for topical administration can be, for example, creams, lotions, eyewashes, throat sprays, nasal drops or aerosols.

The doses depend on the desired effect, on the duration of the treatment and on the route of adminstration used; they are generally between 0.2 mg and 4 mg per kg per day, subcutaneously, i.e. 14 to 280 mg per day for an adult.

In general, the physician will determine the suitable dose as a function of the age, of the weight and of all the other factors specific to the subject to be treated.

The invention also relates to the method for survival and growth of motoneurons, which consists in administering, to the patient, a low molecular weight heparin.

The invention also relates to the method for preventing and/or treating motoneuron diseases, and in particular amyotrophic lateral sclerosis, progressive spinal muscular atrophy, infantile muscular atrophy and primary lateral sclerosis, which consists in administering, to the patient, a low molecular weight heparin.

The invention also relates to the process for preparing medicinal products which are useful for the survival and/or growth of motoneurons, and in particular in the prevention and/or treatment of motoneuron diseases, and in particular amyotrophic lateral sclerosis, progressive spinal muscular atrophy, infantile muscular atrophy and primary lateral sclerosis, consisting in mixing a low molecular pea heparin with one or more compatible and pharmaceutically acceptable diluents and/or adjuvants.

1. Use of a low molecular weight heparin for preparing a medicinal product which is useful for the survival and/or growth of motoneurons.

2. Use, according to claim 1, of a low molecular weight heparin for preparing a medicinal product for the prevention and/or treatment of motoneuron diseases.

3. Use according to claim 2, for the prevention and/or treatment of amyotrophic lateral sclerosis, progressive spinal muscular atrophy, infantile muscular atrophy and lateral sclerosis.

4. Use according to one of claim 1, for which the low molecular weight heparin has a mean molecular weight of between 1 000 and 10 000 daltons.

5. Use according to one of claim 1, for which the low molecular weight heparin has a mean molecular weight of between 1 500 and 6 000 daltons.

6. Use according to one of claim 1, for which the low molecular weight heparin has a mean molecular weight of between 4 000 and 5 000 daltons.

7. Use according to one of claim 1, for which the low molecular weight heparin consists of oligosaccharides having a 2-O-sulfo-4-enopyranosuronic acid at one of their ends.

8. Use according to one of claim 1, for which the low molecular weight heparin is obtained by depolymerization of a heparin ester using a base.

9. Use according to one of claim 1, for which the low molecular weight heparin is enoxaparin.

10. Use according to one of claim 1, for which the low molecular weight heparin is nadroparin.

11. Use according to one of claim 1, for which the low molecular weight heparin is parnaparin.

12. Use according to one of claim 1, for which the low molecular weight heparin is reviparin.

13. Use according to one of claim 1, for which the low molecular weight heparin is dalteparin.

14. Use according to one of claim 1, for which the low molecular weight heparin is tinzaparin.

15. Use according to one of claim 1, for which the low molecular weight heparin is danaparoid.

16. Use according to one of claim 16, for which the low molecular weight heparin is ardeparin.

17. Use according to one of claim 1, for which the low molecular weight heparin is certoparin.

18. Use according to one of claim 1, for which the low molecular weight heparin is CY222.

19. Use according to one of claim 1, for which the low molecular weight heparin is SR90107/ORG31540.
